(19) United States
(12) Patent Application Publication
(10) Pub. No.: US 2018/0008718 A1
(43) Pub. Date: Jan. 11, 2018

(54) COMPOSITIONS FOR THE SUSTAINED RELEASE OF ANTI-GLAUCOMA AGENTS TO CONTROL INTRAOCULAR PRESSURE

(71) Applicants: The Johns Hopkins University, Baltimore, MD (US); Duke University, Durham, NC (US)

(72) Inventors: Jie Fu, Towson, MD (US); Justin Hanes, Baltimore, MD (US); Molly Walsh, Chapel Hill, NC (US); David Epstein, New York, NY (US)

(73) Assignees: The Johns Hopkins University, Baltimore, MD (US); Duke University, Durham, NC (US)

(21) Appl. No.: 15/545,020

(22) PCT Filed: Jan. 19, 2016
(86) PCT No.: PCT/US2016/013914

§ 371 Date: Jul. 20, 2017

Related U.S. Application Data
(60) Provisional application No. 62/105,535, filed on Jan. 20, 2015.

(51) Int. Cl. C08G 65/334; A61K 31/198; A61K 9/00

(52) U.S. Cl. CPC A61K 47/60; C08G 65/3348; A61K 47/6935; A61K 9/0048; A61K 31/198

(57) ABSTRACT

Controlled release dosage formulations for the delivery of active agents, especially for treatment of eye diseases or disorders, such as glaucoma, have been developed. These provide release of the active agent, such as ECA or a derivative thereof, for an effective period of time.

### CROSS-REFERENCE TO RELATED APPLICATIONS

This application claims benefit of U.S. Provisional Application No. 62/105,535, filed Jan. 20, 2015, which is hereby incorporated herein by reference in its entirety.

### FIELD OF THE INVENTION

The present invention relates to polymeric controlled release formulations for the delivery of an effective amount of one or more anti-glaucoma agent, particularly those agents that lower intraocular pressure (IOP), such as ethacrynic acid (ECA) or a derivative thereof to the eye, as well as methods of use thereof for the treatment and prevention of ocular diseases characterized by increased intraocular pressure, such as glaucoma.

### BACKGROUND OF THE INVENTION

Glaucoma is a devastating disease most often associated with elevated intraocular pressure (IOP), induced by the dysfunction of the trabecular meshwork (TM), the tissue responsible for the majority of aqueous humor outflow from the anterior chamber. Elevated IOP causes degeneration of retinal ganglion cells (RGC), resulting in visual field loss and potentially blindness.

Glaucoma affects over 70 million people worldwide and is considered a significant unmet medical need. Current therapies are focused on decreasing intraocular pressure (IOP), which reduces RGC cell degeneration and slows disease progression, even in normal-tension glaucoma. In most patients, IOP lowering agents are delivered topically with eye drops. However, noncompliance with eye drop administration, especially in older patients, is a major issue in glaucoma treatment. Within the next 15 years it is estimated that the glaucoma population will increase by 50% in the United States. Therefore, the identification and development of improved therapeutics and ocular delivery methods to achieve sustained IOP normalization for the treatment of glaucoma is a significant unmet need.

The ideal therapeutic to reduce IOP would be an agent that specifically targets the TM, as 80-90% of aqueous humor outflow occurs through the TM and Schlemms canal. Current commercially available agents, such as timolol, a β-adrenergic receptor antagonist, and latanoprost, a prostaglandin analog, do not target the TM. Timolol functions to decrease aqueous humor production, and can have unwanted systemic respiratory and cardiac effects. Latanoprost, a prostaglandin analog, increases outflow through the uveoscleral pathway, and is responsible for only 3-35% of total aqueous humor outflow. In view of these limitations, multidrug therapy is often necessary to sufficiently lower IOP.

Ethacrynic acid (ECA), FDA approved as a systemically delivered diuretic, works directly on the TM and Schlemms canal to modulate the cellular cytoskeleton and cause cell relaxation in these tissues. ECA has been demonstrated to increase anterior chamber outflow in living monkeys, calf eyes, and cultured human eyes, and decrease IOP in living normal and glaucomatous monkey eyes and in human patients with glaucoma. However, the use of ECA as a topical therapy has been hindered due to its poor ocular penetration, poor distribution to the aqueous humor, and external ocular side effects, caused, at least in part, by its binding to free thiol groups.

ECA toxicity can be reduced by using an ECA-cysteine conjugate which does not affect IOP lowering ability. Therefore, ECA is a promising therapeutic candidate that works directly on the TM to lower IOP. However, the limitations of current therapies, such as patient compliance still exist. Accordingly, there is a need for improved ECA formulations for sustained and slow release of ECA over time, and for delivery methods that display improved ocular safety and physiochemical properties.

Therefore it is an object of the invention to provide formulations containing one or more anti-glaucoma agents, particularly those agents that lower intraocular pressure (IOP), such as ethacrynic acid (ECA) or a derivative thereof and methods of making and using thereof that exhibit improved ocular safety and physiochemical properties.

### SUMMARY OF THE INVENTION

Formulations for the controlled delivery of one or more anti-glaucoma agents, particularly those agents that lower intraocular pressure (IOP), such as ethacrynic acid (ECA) or a derivative thereof conjugated to or dispersed in a polymeric matrix are described herein. The polymeric matrix can be formed from non-biodegradable or biodegradable polymers; however, the polymer matrix is preferably biodegradable. The polymeric matrix can be formed into implants (e.g., rods, disks, wafers, etc.), microparticles, nanoparticles, or combinations thereof for delivery. Upon administration, the agent is released over an extended period of time, either upon degradation of the polymer matrix, diffusion of the one or more inhibitors out of the polymer matrix, or a combination thereof. By employing a polymer-drug conjugate, particles can be formed with more controlled drug loading and drug release profiles. In addition, the solubility of the conjugate can be controlled so as to minimize soluble drug concentration and, therefore, toxicity.

In preferred embodiments, the agent or agents is covalently bound to a polymer, forming a polymer-drug conjugate. The polymer-drug conjugates can then be formed into implants (e.g., rods, wafers, discs, etc.), microparticles, nanoparticles, or combinations thereof for delivery to the eye. By employing a polymer-drug conjugate, particles can be formed with more controlled drug loading and drug release profiles. In addition, the solubility of the conjugate can be controlled by modifying the solubility of the polymer portion and/or the branched point (“Y” in the chemical structure of the polymer, so as to minimize soluble drug concentration and, therefore, toxicity).

In certain embodiments, the polymer-drug conjugates are block copolymers containing ECA or derivative thereof covalently bonded to the block copolymer. In one embodiment, the conjugate has the formula:

(A—X)m-Y—((Z)o-(X)p-(A)q)n

wherein

A represents, independently for each occurrence, one or more anti-glaucoma agents, particularly those agents that lower intraocular pressure (IOP), such as ethacrynic acid (ECA) or a derivative thereof;

X represents, independently for each occurrence, a hydrophobic polymer segment;

Y is absent or represents a branch point;

Z represents, independently for each occurrence, a hydrophilic polymer segment;

o, p, and q are independent 0 or 1;

m represents the number of A-X branches and is an integer between one and twenty; and

n represent the number of Z, Z—X, and Z—X-A branches and is an integer between zero and twenty, more preferably between one and twenty.

Exemplary polymer-drug conjugates of this type are represented by the general formulae shown below

(A-XₘYZ)ₙ

(A-XₘYZ—X)ₙ

(A-XₘYZ—X-A)ₙ

wherein

A represents, independently for each occurrence, one or more anti-glaucoma agents, particularly those agents that lower intraocular pressure (IOP), such as ethacrynic acid (ECA) or a derivative thereof;

X represents, independently for each occurrence, a hydrophobic polymer segment;

Y is absent or represents a branch point;

Z represents, independently for each occurrence, a hydrophilic polymer segment;

m represents the number of A-X branches and is an integer between one and twenty; and

n represent the number of Z, Z—X, and Z—X-A branches and is an integer between zero and twenty, more preferably between one and twenty.

The one or more hydrophobic polymer segments can be any biocompatible, hydrophobic polymer or copolymer. In some cases, the hydrophobic polymer or copolymer is biodegradable. Examples of suitable hydrophobic polymers include, but are not limited to, polyesters such as polylactic acid, polyglycolic acid, or polycaprolactone, polyanhydrides, such as polysebacic anhydride, and copolymers of any of the above. In preferred embodiments, the hydrophobic polymer is a polyanhydride, such as polysebacic anhydride or a copolymer thereof.

The degradation profile of the one or more hydrophobic polymer segments may be selected to influence the release rate of the active agent in vivo. For example, the hydrophobic polymer segments can be selected to degrade over a time period from seven days to 2 years, more preferably from seven days to 56 weeks, more preferably from four weeks to 56 weeks, most preferably from eight weeks to 28 weeks.

The one or more hydrophilic polymer segments can be any hydrophilic, biocompatible, non-toxic polymer or copolymer. In certain embodiments, the one or more hydrophilic polymer segments contain a poly(alkylene glycol), such as polyethylene glycol (PEG). In particular embodiments, the one or more hydrophilic polymer segments are linear PEG chains.

In some embodiments, where both hydrophobic and hydrophilic polymer segments are present, the combined weight average molecular weight of the one or more hydrophilic polymer segments will preferably be larger than the weight average molecular weight of the hydrophobic polymer segment. In some cases, the combined weight average molecular weight of the hydrophilic polymer segments is at least five times, more preferably at least ten times, most preferably at least fifteen times, greater than the weight average molecular weight of the hydrophobic polymer segment.

The branch point, when present, can be an organic molecule which contains three or more functional groups. Preferably, the branch point will contain at least two different types of functional groups (e.g., one or more alcohols and one or more carboxylic acids, or one or more halides and one or more carboxylic acids). In such cases, the different functional groups present on the branch point can be independently addressed synthetically, permitting the covalent attachment of the hydrophobic and hydrophilic segments to the branch point in controlled stoichiometric ratios. In certain embodiments, the branch point is polycarboxylic acid, such as citric acid, tartaric acid, mucic acid, gluconic acid, or 5-hydroxybenzene-1,2,3,-tricarboxylic acid.

In certain embodiments, the polymer-drug conjugate is formed from a single hydrophobic polymer segment and two or more hydrophilic polymer segments covalently connected via a multivalent branch point.

Exemplary polymer-drug conjugates of this type are represented by the general formula shown below

A-X—YZ)ₙ

wherein

A represents one or more anti-glaucoma agents, particularly those agents that lower intraocular pressure (IOP), such as ethacrynic acid (ECA) or a derivative thereof;

X represents a hydrophobic polymer segment;

Y represents a branch point;

Z represents, independently for each occurrence, a hydrophilic polymer segment; and

n is an integer between two and ten.

In certain embodiments, the hydrophilic polymer segments contain a poly(alkylene glycol), such as polyethylene glycol (PEG), preferably linear PEG chains. In some embodiments, the conjugates contain between two and six hydrophilic polymer segments.

In preferred embodiments, the hydrophobic polymer is a polyanhydride, such as polysebacic anhydride or a copolymer thereof. In certain embodiments, the hydrophobic polymer segment is poly(1,6-bis(p-carboxyphenoxy)hexane-co-sebacic acid) (poly(CPH-SA) or poly(1,3-bis(p-carboxyphenoxy)propane-co-sebacic acid) (poly(CPP-SA).

In some embodiments, the branch point connects a single hydrophobic polymer segment to three hydrophilic polyethylene glycol polymer segments. In certain cases, the polymer-drug conjugate can be represented by Formula I

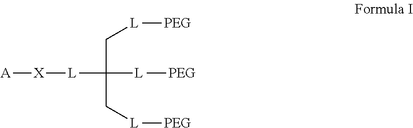

wherein

A is one or more anti-glaucoma agents, particularly those agents that lower intraocular pressure (IOP), such as ethacrynic acid (ECA) or a derivative thereof;

L represents, independently for each occurrence, an ether (e.g., —O—), thioether (e.g., —S—), secondary amine (e.g., —NH—), tertiary amine (e.g., —NR—), secondary amide (e.g., —NHCO—; —CONH—), tertiary amide (e.g., —NRCO—; —CONR—), secondary carbamate (e.g., —OCONH—; —NHCOO—), tertiary carbamate (e.g., —OCONR—; —NRCOO—), urea (e.g., —NHCONH—; —NRCONH—; —NHCONR—, —NRCONR—), sulfinyl group (e.g., —SO—), or sulfonyl group (e.g., —SOO—);

R is, individually for each occurrence, an alkyl, cycloalkyl, heterocycloalkyl, alkylaryl, alkenyl, alkynyl, aryl, or heteroaryl group, optionally substituted with between one and five substituents individually selected from alkyl, cyclopropyl, cyclobutyl ether, amine, halogen, hydroxyl, ether, nitrile, CF3, ester, amide, urea, carbamate, thioether, carboxylic acid, and aryl;

PEG represents a polyethylene glycol chain; and

X represents a hydrophobic polymer segment.

In certain embodiments, the branch point is a citric acid molecule, and the hydrophilic polymer segments are polyethylene glycol. In such cases, the polymer-drug conjugate can be represented by Formula IA:

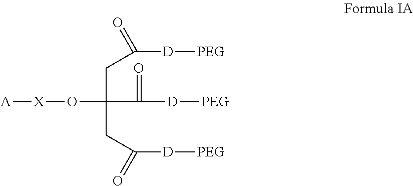

wherein

A is one or more anti-glaucoma agents, particularly those agents that lower intraocular pressure (IOP), such as ethacrynic acid (ECA) or a derivative thereof;

D represents, independently for each occurrence, O or NH;

PEG represents a polyethylene glycol chain; and

X is represents a hydrophobic polymer segment.

X may be any biocompatible hydrophobic polymer or copolymer. In preferred embodiments, the hydrophobic polymer or copolymer is biodegradable. In preferred embodiments, the hydrophobic polymer is a polyanhydride, such as polysebacic anhydride, or a copolymer thereof. The polymer-drug conjugates can be used to form implants (e.g., rods, discs, wafers, etc.), nanoparticles, or microparticles with improved properties for controlled delivery of the one or more agents.

Also provided are pharmaceutical compositions containing implants (e.g., rods, discs, wafers, etc.), nanoparticles, microparticles, or combinations thereof for the controlled release of the agent or agents in combination with one or more pharmaceutically acceptable excipients. The nanoparticles, microparticles, or combination thereof can be formed from one or more polymer-drug conjugates, or blends of polymer-drug conjugates with one or more polymers. The implants (e.g., rods, discs, wafers, etc.), nanoparticles, microparticles, or combination thereof can also be formed from a polymeric matrix having the agent or agents thereof dispersed or encapsulated therein.

The pharmaceutical compositions can be administered to treat or prevent an ocular disease or disorder associated with increased ocular pressure. Upon administration, the agent or agents is released over an extended period of time at concentrations which are high enough to produce therapeutic benefit, but low enough to avoid unacceptable levels of cytotoxicity, and which provide much longer release than inhibitor without conjugate.

### BRIEF DESCRIPTION OF THE DRAWINGS

FIG. 1 is a graph showing the release of ECA-L-cysteine (% release) as a function of time (days).

FIG. 2A is a graph showing intraocular pressure (IOP, mmHg) as function of administration of free ECA and a control over time (days).

FIG. 2B is a graph showing intraocular pressure (IOP, mmHg) as function of administration of ECA-L-cysteine particles and a control over time (days).

### DETAILED DESCRIPTION OF THE INVENTION

### I. Definitions

“Effective amount” or “therapeutically effective amount”, as used herein, refers to an amount of polymer-drug conjugate effective to alleviate, delay onset of, or prevent one or more symptoms of a disease or disorder. In the case of glaucoma, the effective amount of the polymer-drug conjugate reduces intraocular pressure (IOP).

“Biocompatible” and “biologically compatible”, as used herein, generally refer to materials that are, along with any metabolites or degradation products thereof, generally non-toxic to the recipient, and do not cause any significant adverse effects to the recipient. Generally speaking, biocompatible materials are materials which do not elicit a significant inflammatory or immune response when administered to a patient.

“Biodegradable Polymer” as used herein, generally refers to a polymer that will degrade or erode by enzymatic action or hydrolysis under physiologic conditions to smaller units or chemical species that are capable of being metabolized, eliminated, or excreted by the subject. The degradation time is a function of polymer composition, morphology, such as porosity, particle dimensions, and environment.

“Hydrophilic,” as used herein, refers to the property of having affinity for water. For example, hydrophilic polymers (or hydrophilic polymer segments) are polymers (or polymer segments) which are primarily soluble in aqueous solutions and/or have a tendency to absorb water. In general, the more hydrophilic a polymer is, the more that polymer tends to dissolve in, mix with, or be wetted by water.

“Hydrophobic,” as used herein, refers to the property of lacking affinity for, or even repelling water. For example, the more hydrophobic a polymer (or polymer segment), the more that polymer (or polymer segment) tends to not dissolve in, not mix with, or not be wetted by water.

Hydrophilicity and hydrophobicity can be spoken of in relative terms, such as, but not limited to, a spectrum of hydrophilicity/hydrophobicity within a group of polymers or polymer segments. In some embodiments wherein two or more polymers are being discussed, the term “hydrophobic polymer” can be defined based on the polymer's relative hydrophobicity when compared to another, more hydrophilic polymer.

“Nanoparticle”, as used herein, generally refers to a particle having a diameter, such as an average diameter, from about 10 nm up to but not including about 1 micron, preferably from 100 nm to about 1 micron. The particles can have any shape. Nanoparticles having a spherical shape are generally referred to as “nanospheres”.

“Microparticle”, as used herein, generally refers to a particle having a diameter, such as an average diameter, from about 1 micron to about 100 microns, preferably from about 1 to about 50 microns, more preferably from about 1 to about 30 microns, most preferably from about 1 micron to about 10 microns. The microparticles can have any shape. Microparticles having a spherical shape are generally referred to as “microspheres”.

“Molecular weight” as used herein, generally refers to the relative average chain length of the bulk polymer, unless otherwise specified. In practice, molecular weight can be estimated or characterized using various methods including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

“Mean particle size” as used herein, generally refers to the statistical mean particle size (diameter) of the particles in a population of particles. The diameter of an essentially spherical particle may refer to the physical or hydrodynamic diameter. The diameter of a non-spherical particle may refer preferentially to the hydrodynamic diameter. As used herein, the diameter of a non-spherical particle may refer to the largest linear distance between two points on the surface of the particle. Mean particle size can be measured using methods known in the art, such as dynamic light scattering.

“Monodisperse” and “homogeneous size distribution”, are used interchangeably herein and describe a population of nanoparticles or microparticles where all of the particles are the same or nearly the same size. As used herein, a monodisperse distribution refers to particle distributions in which 90% or more of the distribution lies within 15% of the median particle size, more preferably within 10% of the median particle size, most preferably within 5% of the median particle size.

“Pharmaceutically Acceptable”, as used herein, refers to compounds, carriers, excipients, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Branch point”, as used herein, refers to a portion of a polymer-drug conjugate that serves to connect one or more hydrophilic polymer segments to one or more hydrophobic polymer segments.

“Implant,” as generally used herein, refers to a polymeric device or element that is structured, sized, or otherwise configured to be implanted, preferably by injection or surgical implantation, in a specific region of the body so as to provide therapeutic benefit by releasing an active agent such as a glaucoma treating agent like ECA or a derivative thereof over an extended period of time at the site of implantation. For example, intraocular implants are polymeric devices or elements that are structured, sized, or otherwise configured to be placed in the eye, preferably by injection or surgical implantation, and to treat one or more diseases or disorders of the eye by releasing the active agent over an extended period. Intraocular implants are generally biocompatible with physiological conditions of an eye and do not cause adverse side effects. Generally, intraocular implants may be placed in an eye without disrupting vision of the eye.

Ranges of values defined herein include all values within the range as well as all sub-ranges within the range. For example, if the range is defined as an integer from 0 to 10, the range encompasses all integers within the range and any and all subranges within the range, e.g., 1-10, 1-6, 2-8, 3-7, 3-9, etc.

### II. Polymer-ECA Conjugates

Controlled release conjugates containing one or more anti-glaucoma agent, particularly those agents that lower intraocular pressure (IOP), such as ethacrynic acid (ECA) or a derivative thereof conjugated to or dispersed in a polymeric matrix for controlled release of the agent or agents are provided. By administering controlled release conjugates of the agent or agents, activity is enhanced and prolonged while toxicity is reduced or eliminated.

In some embodiments, the agent or agent is dispersed or encapsulated in a polymeric matrix for delivery to the eye. The polymeric matrix can be formed from non-biodegradable or biodegradable polymers; however, the polymer matrix is preferably biodegradable. The polymeric matrix can be formed into implants, microparticles, nanoparticles, or combinations thereof for delivery to the eye. Upon administration, the agent or agents is released over an extended period of time, either upon degradation of the polymer matrix, diffusion of the one or more inhibitors out of the polymer matrix, or a combination thereof. By employing a polymer-drug conjugate, particles can be formed with more controlled drug loading and drug release profiles.

In some embodiments, the controlled-release formulation contains particles formed from one or more polymer-drug conjugates. The polymer-drug conjugates are block copolymers containing the agent or agents covalently bonded to the block copolymer. Typically, the polymer-drug conjugates contain the agent or agents, one or more hydrophobic polymer segments, and one or more hydrophilic polymer segments. In certain cases, one or more hydrophilic polymer segments are attached to the one or more hydrophobic polymer segments by a branch point. By employing a polymer-drug conjugate, particles can be formed with more controlled drug loading and drug release profiles. In addition, the solubility of the conjugate can be controlled so as to minimize soluble drug concentration and, therefore, toxicity.

A. Polymers

Hydrophobic Polymers

Polymer-drug conjugates can contain one or more hydrophobic polymer segments. The hydrophobic polymer segments can be homopolymers or copolymers.

In preferred embodiments, the hydrophobic polymer segment is a biodegradable polymer. In cases where the hydrophobic polymer is biodegradable, the polymer degradation profile may be selected to influence the release rate of the active agent in vivo. For example, the hydrophobic polymer segment can be selected to degrade over a time period from seven days to 2 years, more preferably from seven days to 56 weeks, more preferably from four weeks to 56 weeks, most preferably from eight weeks to 28 weeks.

Examples of suitable hydrophobic polymers include polyhydroxyacids such as poly(lactic acid), poly(glycolic acid), and poly(lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(hydroxyalkanoates); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof.

In preferred embodiments, the hydrophobic polymer segment is a polyanhydride. The polyanhydride can be an aliphatic polyanhydride, an unsaturated polyanhydride, or an aromatic polyanhydride. Representative polyanhydrides include polyadipic anhydride, polyfumaric anhydride, polysebacic anhydride, polymaleic anhydride, polymalic anhydride, polyphthalic anhydride, polyisophthalic anhydride, polyaspartic anhydride, polyterephthalic anhydride, polyisophthalic anhydride, poly carboxyphenoxypropane anhydride, polycarboxyphenoxyhexane anhydride, as well as copolymers of these polyanhydrides with other polyanhydrides at different mole ratios. Other suitable polyanhydrides are disclosed in U.S. Pat. Nos. 4,757,128, 4,857,311, 4,888,176, and 4,789,724. The polyanhydride can also be a copolymer containing polyanhydride blocks.

In certain embodiments, the hydrophobic polymer segment is polysebacic anhydride. In certain embodiments, the hydrophobic polymer segment is poly(1,6-bis(p-carboxyphenoxy)hexane-co-sebacic acid) (poly(CPH-SA). In certain embodiments, the hydrophobic polymer segment is poly(1,3-bis(p-carboxyphenoxy)propane-co-sebacic acid) (poly(CPP-SA).

The molecular weight of the hydrophobic polymer can be varied to prepare polymer-drug conjugates that form particles having properties, such as drug release rate, optimal for specific applications. The hydrophobic polymer segment can have a molecular weight of about 150 Da to 1 MDa. In certain embodiments, the hydrophobic polymer segment has a molecular weight of between about 1 kDa and about 100 kDa, more preferably between about 1 kDa and about 50 kDa, most preferably between about 1 kDa and about 25 kDa.

In some cases, the hydrophobic polymer segment has a molecular weight which is less that the average molecular weight of the one or more hydrophilic polymer segments of the polymer-drug conjugate. In a preferred embodiment, the hydrophobic polymer segment has a molecular weight of less than about 5 kDa.

Hydrophilic Polymers

Polymer-drug conjugates can also contain one or more hydrophilic polymer segments. The one or more hydrophilic polymer segments can be any hydrophilic, biocompatible, non-toxic polymer or copolymer. Preferably, the polymer-drug conjugates contain more than one hydrophilic polymer segment. In some embodiments, the polymer-drug conjugate contains between two and six, more preferably between three and five, hydrophilic polymer segments. In certain embodiments, the polymer drug conjugate contains three hydrophilic polymer segments.

Each hydrophilic polymer segment can independently be any hydrophilic, biocompatible (i.e., it does not induce a significant inflammatory or immune response), non-toxic polymer or copolymer. Examples of suitable polymers include, but are not limited to, poly(alkylene glycols) such as polyethylene glycol (PEG), poly(propylene glycol) (PPG), and copolymers of ethylene glycol and propylene glycol, poly(oxyethylated polyol), poly(olefinic alcohol), polyvinylpyrrolidone), poly(hydroxyalkylmethacrylamide), poly(hydroxyalkylmethacrylate), poly(saccharides), poly(amino acids), poly(hydroxy acids), poly(vinyl alcohol), and copolymers, terpolymers, and mixtures thereof.

In preferred embodiments, the one or more hydrophilic polymer segments contain a poly(alkylene glycol) chain. The poly(alkylene glycol) chains may contain between 8 and 500 repeat units, more preferably between 40 and 500 repeat units. Suitable poly(alkylene glycols) include polyethylene glycol), polypropylene 1,2-glycol, poly(propylene oxide), polypropylene 1,3-glycol, and copolymers thereof. In certain embodiments, the one or more hydrophilic polymer segments are PEG chains. In such cases, the PEG chains can be linear or branched, such as those described in U.S. Pat. No. 5,932,462. In certain embodiments, the PEG chains are linear.

Each of the one or more hydrophilic polymer segments can independently have a molecular weight of about 300 Da to 1 MDa. The hydrophilic polymer segment may have a molecular weight ranging between any of the molecular weights listed above. In certain embodiments, each of the one or more hydrophilic polymer segments has a molecular weight of between about 1 kDa and about 20 kDa, more preferably between about 1 kDa and about 15 kDa, most preferably between about 1 kDa and about 10 kDa. In a preferred embodiment, each of the one or more hydrophilic polymer segments has a molecular weight of about 5 kDa. In cases where both hydrophobic and hydrophilic polymer segments are present, the combined molecular weight of the one or more hydrophilic polymer segments will preferably be larger than the molecular weight of the hydrophobic polymer segment. In some cases, the combined molecular weight of the hydrophilic polymer segments is at least five times, more preferably at least ten times, most preferably at least fifteen times, greater than the molecular weight of the hydrophobic polymer segment.

Branch Points

The functional groups may be any atom or group of atoms that contains at least one atom that is neither carbon nor hydrogen, with the proviso that the groups must be capable of reacting with the hydrophobic and hydrophilic polymer segments. Suitable functional groups include halogens (bromine, chlorine, and iodine); oxygen-containing functional groups such as a hydroxyls, epoxides, carbonyls, aldehydes, ester, carboxyls, and acid chlorides; nitrogen-containing functional groups such as amines and azides; and sulfur-containing groups such as thiols. The functional group may also be a hydrocarbon moiety which contains one or more non-aromatic pi-bonds, such as an alkyne, alkene, or diene. Preferably, the branch point will contain at least two different types of functional groups (e.g., one or more alcohols and one or more carboxylic acids, or one or more halides and one or more alcohols). In such cases, the different functional groups present on the branch point can be independently addressed synthetically, permitting the covalent attachment of the hydrophobic and hydrophilic segments to the branch point in controlled stoichiometric ratios.

The branch point, when present, can be an organic molecule which contains three or more functional groups. Preferably, the branch point will contain at least two different types of functional groups (e.g., one or more alcohols and one or more carboxylic acids, or one or more halides and one or more carboxylic acids or one or more amines)). In such cases, the different functional groups present on the branch point can be independently addressed synthetically, permitting the covalent attachment of the hydrophobic and hydrophilic segments to the branch point in controlled stoichiometric ratios. In certain embodiments, the branch point is polycarboxylic acid, such as citric acid, tartaric acid, mucic acid, gluconic acid, or 5-hydroxybenzene-1,2,3,-tricarboxylic acid.

Following reaction of the hydrophobic and hydrophilic polymer segments with functional groups on the branch point, the one or more hydrophobic polymer segments and the one or more hydrophilic polymer segments will be covalently joined to the branch point via linking moieties. The identity of the linking moieties will be determined by the identity of the functional group and the reactive locus of the hydrophobic and hydrophilic polymer segments (as these elements react to form the linking moiety or a precursor of the linking moiety). Examples of suitable linking moieties that connect the polymer segments to the branch point include secondary amides (—CONH—), tertiary amides (—CONR—), secondary carbamates (—OCONH—; —NHCOO—), tertiary carbamates (—OCONR—; —NRCOO—), ureas (—NHCONH—; —NRCONH—; —NHCONR—, —NRCONR—), carbinols (—CHOH—, —CROH—), ethers (—O—), and esters (—COO—, —CH₂O₂C—, CHRO₂C—), wherein R is an alkyl group, an aryl group, or a heterocyclic group. In certain embodiments, the polymer segments are connected to the branch point via an ester (—COO—, —CH₂O₂C—, CHRO₂C—), a secondary amide (—CONH—), or a tertiary amide (—CONR—), wherein R is an alkyl group, an aryl group, or a heterocyclic group.

In certain embodiments, the branch point is polycarboxylic acid, such as citric acid, tartaric acid, mucic acid, gluconic acid, or 5-hydroxybenzene-1,2,3,-tricarboxylic acid. Exemplary branch points include the following organic compounds:

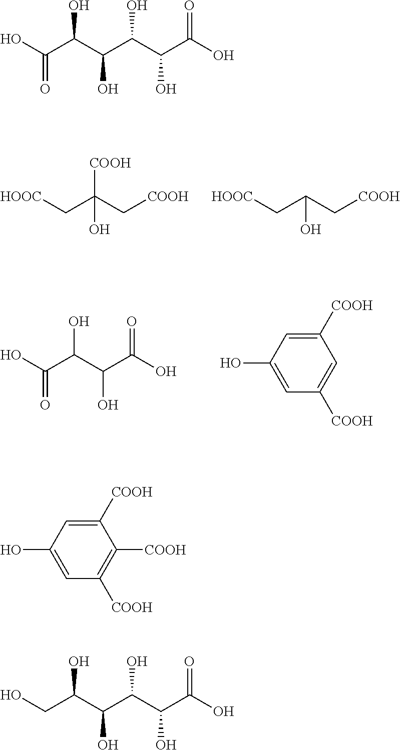

In certain embodiments, the polymer-drug conjugate contains the agent or agents covalently attached to a bioerodible polymeric segment. Preferably, the bioerodible segment to which the agent or agents is attached is composed of one or more monomers that possess low solubility in aqueous solution. In certain embodiments, one or more of the monomers possesses a solubility of less than 2 g/L, more preferably less than 1 g/L, more preferably less than 0.5 g/L, most preferably less than 0.3 g/L in water.

B. Therapeutic, Prophylactic or Diagnostic Agent

The conjugates may have bound thereto a therapeutic, prophylactic or diagnostic agent. Preferably, polymer-drug conjugates contain one or more anti-glaucoma agents, particularly those agents that lower intraocular pressure (IOP), such as ethacrynic acid (ECA) or a derivative thereof covalently attached to a block copolymer.

The formulations/conjugates contain one or more anti-glaucoma agents. In some embodiments, the one or more agents treat glaucoma by lowering intraocular pressure (IOP). In particular embodiments, the one or more agents lower IOP by acting directly on the trabecular meshwork (TM).

Representative anti-glaucoma agents include prostaglandin analogs (such as travoprost, bimatoprost, and latanoprost), beta-adrenergic receptor antagonists (such as timolol, betaxolol, levobetaxolol, and carteolol), alpha-2 adrenergic receptor agonists (such as brimonidine and apraclonidine), carbonic anhydrase inhibitors (such as brinzolamide, acetazolamine, and dorzolamide), miotics (i.e., parasympathomimetics, such as pilocarpine and ecothiopate), seretonergics muscarinics, dopaminergic agonists, and adrenergic agonists (such as apraclonidine and brimonidine).

In one embodiment, the conjugate has the formula:

(A-X)ₘ—Y—((Z)ₒ—(X)ₚ-(A)_{q})ₙ

wherein

A represents, independently for each occurrence, one or more anti-glaucoma agents, particularly those agents that lower intraocular pressure (IOP), such as ethacrynic acid (ECA) or a derivative thereof;

X represents, independently for each occurrence, a hydrophobic polymer segment;

Y is absent or represents a branch point;

Z represents, independently for each occurrence, a hydrophilic polymer segment;

o, p, and q are independent 0 or 1;

m represents the number of A-X branches and is an integer between one and twenty; and

n represent the number of Z, Z—X, and Z—X-A branches and is an integer between zero and twenty, more preferably between one and twenty.

Exemplary polymer-drug conjugates are represented by the general formulae shown below:

(A-XₘYZ)ₙ

(A-XₘYZ—X)ₙ

(A-XₘYZ—X-A)ₙ

wherein

A represents, independently for each occurrence, one or more anti-glaucoma agents, particularly those agents that lower intraocular pressure (IOP), such as ethacrynic acid (ECA) or a derivative thereof;

X represents, independently for each occurrence, a hydrophobic polymer segment;

Y is absent, or represents a branch point;

Z represents, independently for each occurrence, a hydrophilic polymer segment; and

m represents the number of A-X branches and is an integer between one and twenty; and

n represents the number of Z, Z—X, and Z—X-A branches and is an integer between zero and twenty, more preferably between one and 20.

In certain embodiments, the polymer-drug conjugate is formed from a single hydrophobic polymer segment and two or more hydrophilic polymer segments covalently connected via a multivalent branch point. Exemplary polymer-drug conjugates of this type are represented by the general formula shown below

A-X—YZ)ₙ

wherein

A represents, independently for each occurrence, one or more anti-glaucoma agents, particularly those agents that lower intraocular pressure (IOP), such as ethacrynic acid (ECA) or a derivative thereof;

X represents, a hydrophobic polymer segment;

Y represents a branch point;

Z represents, independently for each occurrence, a hydrophilic polymer segment; and

n is an integer between zero and 300, more preferably between zero and fifty, more preferably between zero and thirty, most preferably between zero and ten.

In some embodiments, the branch point connects a single hydrophobic polymer segment to three hydrophilic polyethylene glycol polymer segments.

In certain cases, the polymer-drug conjugate can be represented by Formula I

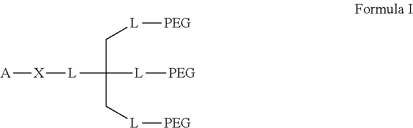

wherein

A is one or more anti-glaucoma agents, particularly those agents that lower intraocular pressure (IOP), such as ethacrynic acid (ECA) or a derivative thereof;

L represents, independently for each occurrence, an ether (e.g., —O—), thioether (e.g., —S—), secondary amine (e.g., —NH—), tertiary amine (e.g., —NR—), secondary amide (e.g., —NHCO—; —CONH—), tertiary amide (e.g., —NRCO—; —CONR—), secondary carbamate (e.g., —OCONH—; —NHCOO—), tertiary carbamate (e.g., —OCONR—; —NRCOO—), urea (e.g., —NHCONH—; —NRCONH—; —NHCONR—, —NRCONR—), sulfinyl group (e.g., —SO—), or sulfonyl group (e.g., —SOO—);

R is, individually for each occurrence, an alkyl, cycloalkyl, heterocycloalkyl, alkylaryl, alkenyl, alkynyl, aryl, or heteroaryl group, optionally substituted with between one and five substituents individually selected from alkyl, cyclopropyl, cyclobutyl ether, amine, halogen, hydroxyl, ether, nitrile, CF₃, ester, amide, urea, carbamate, thioether, carboxylic acid, and aryl;

PEG represents a polyethylene glycol chain; and

X represents a hydrophobic polymer segment.

In certain embodiments, the branch point is a citric acid molecule, and the hydrophilic polymer segments are polyethylene glycol. In such cases, the polymer-drug conjugate can be represented by Formula IA

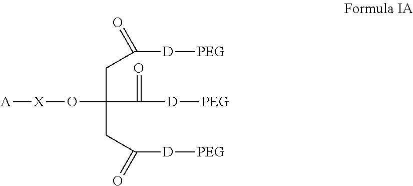

wherein

A is one or more anti-glaucoma agents, particularly those agents that lower intraocular pressure (IOP), such as ethacrynic acid (ECA) or a derivative thereof;

D represents, independently for each occurrence, O or NH;

PEG represents a polyethylene glycol chain; and

X is represents a hydrophobic polymer segment.

In some embodiments, D is, in every occurrence, O. In other embodiments, D is, in every occurrence, NH. In still other embodiments, D is, independently for each occurrence, O or NH.

In some embodiments, the polymer drug conjugate is defined by the following formula

A-X

wherein

A is one or more anti-glaucoma agents, particularly those agents that lower intraocular pressure (IOP), such as ethacrynic acid (ECA) or a derivative thereof; and

X is a hydrophobic polymer segment, preferably a polyanhydride.

In some embodiments, the anti-glaucoma agent is ethacrynic acid or a derivative thereof. Ethacrynic acid a phenoxyacetic acid derivative containing a ketone group and a methylene group. The structure is shown below:

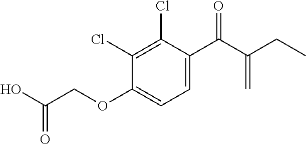 [SMILES: C=C(CC)C(=O)c1ccc(OCC(=O)O)c(Cl)c1Cl]

A cysteine adduct is formed with the methylene group and this is the active form.

Ethacrynic acid can cause low potassium levels, which may manifest as muscle cramps or weakness. It has also been known to cause reversible or permanent hearing loss (ototoxicity) and liver damage when administered in high dosages. On oral administration, it produces diarrhea; intestinal bleeding may occur at higher doses.

ECA, which is FDA approved as a systemically delivered diuretic, works directly on the TM and Schlemms canal to modulate the cellular cytoskeleton and cause cell relaxation in these tissues. ECA has shown to increase anterior chamber outflow in living monkeys, calf eyes, and cultured human eyes, and decrease IOP in living normal and glaucomatous monkey eyes and in human patients with glaucoma. However, the use of ECA as a topical therapy has been hindered due to its poor ocular penetration, poor distribution to the aqueous humor, and external ocular side effects, caused, at least in part, by its binding to free thiol groups.

ECA toxicity can be reduced by using an ECA-cysteine conjugate that does not affect is IOP lowering ability. The structure of the conjugate is shown below:

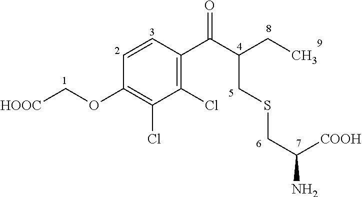 [SMILES: CCC(CSC[C@H](N)C(=O)O)C(=O)c1ccc(OCC(=O)O)c(Cl)c1Cl]

Exemplary polymer-ECA-L-cysteine drug conjugates are shown below:

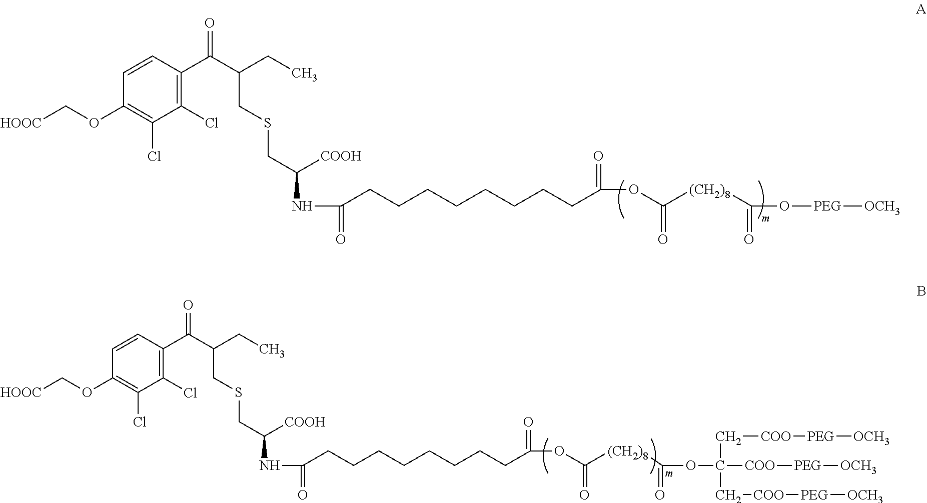

Chemical structure of the ECA-cysteine particles, PEG-SA-ECA-L-Cysteine (A) and, PEG₃-SA-ECA-L-Cysteine (B).

In addition to the one or more anti-glaucoma agents, particularly those agents that lower intraocular pressure (IOP), such as ethacrynic acid (ECA) or a derivative thereof present in the polymeric particles, the formulation can contain one or more additional therapeutic, diagnostic, and/or prophylactic agents. The active agents can be a small molecule active agent or a biomolecule, such as an enzyme or protein, polypeptide, or nucleic acid. Suitable small molecule active agents include organic and organometallic compounds. In some instances, the small molecule active agent has a molecular weight of less than about 2000 g/mol, more preferably less than about 1500 g/mol, most preferably less than about 1200 g/mol. The small molecule active agent can be a hydrophilic, hydrophobic, or amphiphilic compound.

In some cases, one or more additional active agents may be encapsulated in, dispersed in, or otherwise associated with particles formed from one or more polymer-drug conjugates. In certain embodiments, one or more additional active agents may also be dissolved or suspended in the pharmaceutically acceptable carrier.

In the case of pharmaceutical compositions for the treatment of ocular diseases, the formulation may contain one or more ophthalmic drugs. In particular embodiments, the ophthalmic drug is a drug used to treat, prevent or diagnose a disease or disorder of the posterior segment eye. Non-limiting examples of ophthalmic drugs include anti-angiogenesis agents, anti-infective agents, anti-inflammatory agents, growth factors, immunosuppressant agents, anti-allergic agents, and combinations thereof.

Representative anti-angiogenesis agents include, but are not limited to, antibodies to vascular endothelial growth factor (VEGF) such as bevacizumab (AVASTIN®) and rhuFAb V2 (ranibizumab, LUCENTIS®), and other anti-VEGF compounds including aflibercept (EYLEA®); MACUGEN® (pegaptanim sodium, anti-VEGF aptamer or EYE001) (Eyetech Pharmaceuticals); pigment epithelium derived factor(s) (PEDF); COX-2 inhibitors such as celecoxib (CELEBREX®) and rofecoxib (VIOXX®); interferon alpha; interleukin-12 (IL-12); thalidomide (THALOMID®) and derivatives thereof such as lenalidomide (REVLIMID®); squalamine; endostatin; angiostatin; ribozyme inhibitors such as ANGIOZYME® (Sirna Therapeutics); multifunctional antiangiogenic agents such as NEOVASTAT® (AE-941) (Aeterna Laboratories, Quebec City, Canada); receptor tyrosine kinase (RTK) inhibitors such as sunitinib (SUTENT®); tyrosine kinase inhibitors such as sorafenib (Nexavar®) and erlotinib (Tarceva®); antibodies to the epidermal grown factor receptor such as panitumumab (VECTIBIX®) and cetuximab (ERBITUX®), as well as other anti-angiogenesis agents known in the art.

Anti-infective agents include antiviral agents, antibacterial agents, antiparasitic agents, and anti-fungal agents. Representative antiviral agents include ganciclovir and acyclovir. Representative antibiotic agents include aminoglycosides such as streptomycin, amikacin, gentamicin, and tobramycin, ansamycins such as geldanamycin and herbimycin, carbacephems, carbapenems, cephalosporins, glycopeptides such as vancomycin, teicoplanin, and telavancin, lincosamides, lipopeptides such as daptomycin, macrolides such as azithromycin, clarithromycin, dirithromycin, and erythromycin, monobactams, nitrofurans, penicillins, polypeptides such as bacitracin, colistin and polymyxin B, quinolones, sulfonamides, and tetracyclines.

In some cases, the active agent is an anti-allergic agent such as olopatadine and epinastine.

Anti-inflammatory agents include both non-steroidal and steroidal anti-inflammatory agents. Suitable steroidal active agents include glucocorticoids, progestins, mineralocorticoids, and corticosteroids.

The ophthalmic drug may be present in its neutral form, or in the form of a pharmaceutically acceptable salt. In some cases, it may be desirable to prepare a formulation containing a salt of an active agent due to one or more of the salt's advantageous physical properties, such as enhanced stability or a desirable solubility or dissolution profile.

Generally, pharmaceutically acceptable salts can be prepared by reaction of the free acid or base forms of an active agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an active agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, Md., 2000, p. 704. Examples of ophthalmic drugs sometimes administered in the form of a pharmaceutically acceptable salt include timolol maleate, brimonidine tartrate, and sodium diclofenac.

In some cases, the active agent is a diagnostic agent imaging or otherwise assessing the eye. Exemplary diagnostic agents include paramagnetic molecules, fluorescent compounds, magnetic molecules, and radionuclides, x-ray imaging agents, and contrast media.

In certain embodiments, the pharmaceutical composition contains one or more local anesthetics. Representative local anesthetics include tetracaine, lidocaine, amethocaine, proparacaine, lignocaine, and bupivacaine. In some cases, one or more additional agents, such as a hyaluronidase enzyme, is also added to the formulation to accelerate and improves dispersal of the local anesthetic.

### III. Synthesis of Polymer-Drug Conjugates

Polymer-drug conjugates can be prepared using synthetic methods known in the art. Representative methodologies for the preparation of polymer-drug conjugates are discussed below. The appropriate route for synthesis of a given polymer-drug conjugate can be determined in view of a number of factors, such as the structure of the polymer-drug conjugate, the identity of the polymers which make up the conjugate, the identity of the active agent, as well as the structure of the compound as a whole as it relates to compatibility of functional groups, protecting group strategies, and the presence of labile bonds.

In addition to the synthetic methodologies discussed below, alternative reactions and strategies useful for the preparation of the polymer-dug conjugates disclosed herein are known in the art. See, for example, March, “Advanced Organic Chemistry,” 5^{th} Edition, 2001, Wiley-Interscience Publication, New York). Generally, polymer-drug conjugates are prepared by first forming the polymeric component of the polymer-drug conjugate, and then covalently attaching an active agent.

A. ECA-L-Cysteine

ECA-L-cysteine can be prepared using techniques known in the art. For example, ethacrynic acid is added to water and the pH is adjusted to 5.0 until the ethacrynic acid dissolves, after which the pH is adjusted to 7. L-cysteine is dissolved in water and the pH adjusted to 7.0. The two solutions are mixed together with gentle rolling for one hour, after which the solution is lyophilized.

B. PEG₃-PSA (PEG-PSA) prepolymer

In a first step, sebacic acid is refluxed is acetic anhydride to form an acylated polysebacic acid precursor (PreSA). An excess of PreSA is then combined with polyethylene glycol methyl ether, and polymerized under anhydrous hot-melt polymerization conditions. The resulting polymer (PEG-PSA) can then be reacted with ECA-L-cysteine to form the polymer-drug conjugate.

The synthesis of an exemplary polymer-drug conjugate containing multiple hydrophilic polymer segments (three PEG chains) attached to a hydrophobic polymer segment (poly(sebacic anhydride) via a branch point (citric acid) is described in Scheme 1.

In the case of polymer-drug conjugates containing a branch point, synthesis of the polymer drug conjugate will typically begin by sequentially attaching the hydrophobic polymer segment and the hydrophilic polymer segments to the branch point to form the polymeric portion of the polymer-drug conjugate. As shown in scheme 1, citric acid is first reacted with CH₃O-PEG-NH₂ in the presence of N,N′-dicyclohexylcarbodiimide (DCC) and a catalytic amount of 4-dimethylaminopyridine (DMAP), forming amide linkages between the PEG chains and the three carboxylic acid residues of the citric acid branch point. The resulting compound is then reacted with an acylated polysebacic acid precursor (PreSA), and polymerized under anhydrous hot-melt polymerization conditions. The resulting polymer (PEG₃-PSA) is then reacted with ECA-L-cysteine to form the polymer-drug conjugate.

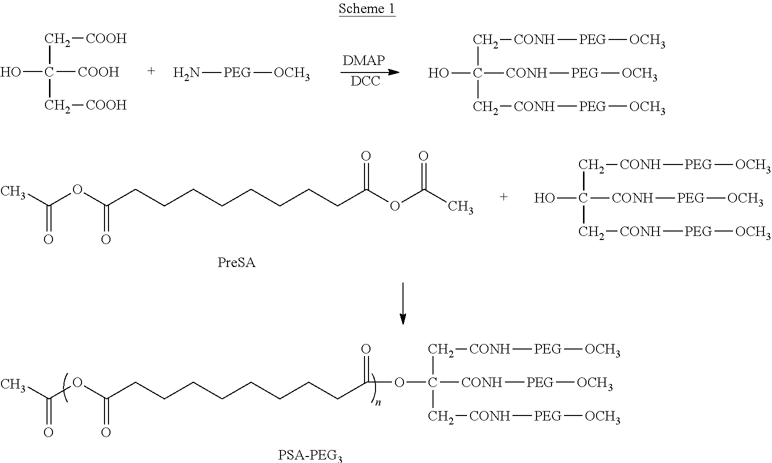

### IV. Particles and Implants for Controlled Delivery of Anti-Glaucoma Agents

Polymeric implants (e.g., rods, discs, wafers, etc.), microparticles, and nanoparticles for the controlled delivery of one or more anti-glaucoma agents, particularly those agents that lower intraocular pressure (IOP), such as ethacrynic acid (ECA) or a derivative thereof are provided, either formed of the conjugates or having the conjugates dispersed or encapsulated in a matrix. In some embodiments, the particles or implants contain the agent or agents dispersed or encapsulated in a polymeric matrix. In preferred embodiments, the particles or implants are formed from polymer-drug conjugates containing the agent or agents which are covalently bound to a polymer.

A. Particles Formed from Polymer-Drug Conjugates

Microparticles and nanoparticles can be formed from one or more species of polymer-drug conjugates. In some cases, particles are formed from a single polymer-drug conjugate (i.e., the particles are formed from a polymer-drug conjugate which contains the same active agent, hydrophobic polymer segment, branch point (when present), and hydrophilic polymer segment or segments).

In other embodiments, the particles are formed from a mixture of two or more different polymer-drug conjugates. For example, particles may be formed from two or more polymer-drug conjugates containing the agent or agents and the same hydrophobic polymer segment, branch point (when present), and hydrophilic polymer segment or segments. In other cases, the particles are formed from two or more polymer-drug conjugates containing the agent or agents, and different hydrophobic polymer segments, branch points (when present), and/or hydrophilic polymer segments. Such particles can be used, for example, to vary the release rate of the agent or agents.

Particles can also be formed from blends of polymer-drug conjugates with one or more additional polymers. In these cases, the one or more additional polymers can be any of the non-biodegradable or biodegradable polymers described in Section B below, although biodegradable polymers are preferred. In these embodiments, the identity and quantity of the one or more additional polymers can be selected, for example, to influence particle stability, i.e. that time required for distribution to the site where delivery is desired, and the time desired for delivery.

Particles having an average particle size of between 10 nm and 1000 microns are useful in the compositions described herein. In preferred embodiments, the particles have an average particle size of between 10 nm and 100 microns, more preferably between about 100 nm and about 50 microns, more preferably between about 200 nm and about 50 microns. In certain embodiments, the particles are nanoparticles having a diameter of between 500 and 700 nm. The particles can have any shape but are generally spherical in shape.

In some embodiments, the population of particles formed from one or more polymer-drug conjugates is a monodisperse population of particles. In other embodiments, the population of particles formed from one or more polymer-drug conjugates is a polydisperse population of particles. In some instances where the population of particles formed from one or more polymer-drug conjugates is polydisperse population of particles, greater that 50%, 60%, 65%, 70%, 75%, 80%, 85%, 90%, or 95% of the particle size distribution lies within 10% of the median particle size.

Preferably, particles formed from one or more polymer-drug conjugates contain significant amounts of a hydrophilic polymer, such as PEG, on their surface.

### Methods of Forming Microparticles and Nanoparticles

Microparticle and nanoparticles can be formed using any suitable method for the formation of polymer micro- or nanoparticles known in the art. The method employed for particle formation will depend on a variety of factors, including the characteristics of the polymers present in the polymer-drug conjugate or polymer matrix, as well as the desired particle size and size distribution.

In circumstances where a monodisperse population of particles is desired, the particles may be formed using a method which produces a monodisperse population of nanoparticles. Alternatively, methods producing polydisperse nanoparticle distributions can be used, and the particles can be separated using methods known in the art, such as sieving, following particle formation to provide a population of particles having the desired average particle size and particle size distribution.

Common techniques for preparing microparticles and nanoparticles include, but are not limited to, solvent evaporation, hot melt particle formation, solvent removal, spray drying, phase inversion, coacervation, and low temperature casting. Suitable methods of particle formulation are briefly described below. Pharmaceutically acceptable excipients, including pH modifying agents, disintegrants, preservatives, and antioxidants, can optionally be incorporated into the particles during particle formation.

### 1. Solvent Evaporation

In this method, the polymer-drug conjugate (or polymer matrix and ECA or a derivative thereof) is dissolved in a volatile organic solvent, such as methylene chloride. The organic solution containing the polymer-drug conjugate is then suspended in an aqueous solution that contains a surface active agent such as poly(vinyl alcohol). The resulting emulsion is stirred until most of the organic solvent evaporated, leaving solid nanoparticles. The resulting nanoparticles are washed with water and dried overnight in a lyophilizer. Nanoparticles with different sizes and morphologies can be obtained by this method.

Polymer-drug conjugates which contain labile polymers, such as certain polyanhydrides, may degrade during the fabrication process due to the presence of water. For these polymers, the following two methods, which are performed in completely anhydrous organic solvents, can be used.

### 2. Hot Melt Particle Formation

In this method, the polymer-drug conjugate (or polymer matrix and ECA or a derivative thereof) is first melted, and then suspended in a non-miscible solvent (like silicon oil), and, with continuous stirring, heated to 5° C. above the melting point of the polymer-drug conjugate. Once the emulsion is stabilized, it is cooled until the polymer-drug conjugate particles solidify. The resulting nanoparticles are washed by decantation with a suitable solvent, such as petroleum ether, to give a free-flowing powder. The external surfaces of particles prepared with this technique are usually smooth and dense. Hot melt particle formation can be used to prepare particles containing polymer-drug conjugates which are hydrolytically unstable, such as certain polyanhydrides. Preferably, the polymer-drug conjugate used to prepare microparticles via this method will have an overall molecular weight of less than 75,000 Daltons.

### 3. Solvent Removal

Solvent removal can also be used to prepare particles from polymer-drug conjugates that are hydrolytically unstable. In this method, the polymer-drug conjugate (or polymer matrix and ECA or a derivative thereof). is dispersed or dissolved in a volatile organic solvent such as methylene chloride. This mixture is then suspended by stirring in an organic oil (such as silicon oil) to form an emulsion. Solid particles form from the emulsion, which can subsequently be isolated from the supernatant. The external morphology of spheres produced with this technique is highly dependent on the identity of the polymer-drug conjugate.

### 4. Spray Drying

In this method, the polymer-drug conjugate (or polymer matrix and ECA or a derivative thereof) is dissolved in an organic solvent such as methylene chloride. The solution is pumped through a micronizing nozzle driven by a flow of compressed gas, and the resulting aerosol is suspended in a heated cyclone of air, allowing the solvent to evaporate from the microdroplets, forming particles. Particles ranging between 0.1-10 microns can be obtained using this method.

### 5. Phase Inversion

Particles can be formed from polymer-drug conjugates using a phase inversion method. In this method, the polymer-drug conjugate (or polymer matrix and ECA or a derivative thereof) is dissolved in a “good” solvent, and the solution is poured into a strong non solvent for the polymer-drug conjugate to spontaneously produce, under favorable conditions, microparticles or nanoparticles. The method can be used to produce nanoparticles in a wide range of sizes, including, for example, about 100 nanometers to about 10 microns, typically possessing a narrow particle size distribution.

### 6. Coacervation

Techniques for particle formation using coacervation are known in the art, for example, in GB-B-929 406; GB-B-929 40 1; and U.S. Pat. Nos. 3,266,987, 4,794,000, and 4,460,563. Coacervation involves the separation of a polymer-drug conjugate (or polymer matrix and ECA or a derivative thereof) solution into two immiscible liquid phases. One phase is a dense coacervate phase, which contains a high concentration of the polymer-drug conjugate, while the second phase contains a low concentration of the polymer-drug conjugate. Within the dense coacervate phase, the polymer-drug conjugate forms nanoscale or microscale droplets, which harden into particles. Coacervation may be induced by a temperature change, addition of a non-solvent or addition of a micro-salt (simple coacervation), or by the addition of another polymer thereby forming an interpolymer complex (complex coacervation).

### 7. Low Temperature Casting

Methods for very low temperature casting of controlled release microspheres are described in U.S. Pat. No. 5,019,400 to Gombotz et al. In this method, the polymer-drug conjugate (or polymer matrix and ECA or a derivative thereof) is dissolved in a solvent. The mixture is then atomized into a vessel containing a liquid non-solvent at a temperature below the freezing point of the polymer-drug conjugate solution which freezes the polymer-drug conjugate droplets. As the droplets and non-solvent for the polymer-drug conjugate are warmed, the solvent in the droplets thaws and is extracted into the non-solvent, hardening the microspheres.

B. Dispersions of Particles Containing One or More Anti-Glaucoma Agents in a Polymer Matrix

Particles can also be formed containing one or more anti-glaucoma agents, particularly those agents that lower intraocular pressure (IOP), such as ethacrynic acid (ECA) or a derivative thereof dispersed or encapsulated in a polymeric matrix, which can be a solid or hydrogel. The matrix can be formed of non-biodegradable or biodegradable matrices, although biodegradable matrices are preferred. The polymer is selected based on the time required for in vivo stability, i.e. that time required for distribution to the site where delivery is desired, and the time desired for delivery.

Representative synthetic polymers are: poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terephthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), copolymers and blends thereof. As used herein, “derivatives” include polymers having substitutions, additions of chemical groups, for example, alkyl, alkylene, hydroxylations, oxidations, and other modifications routinely made by those skilled in the art.

Examples of preferred biodegradable polymers include polymers of hydroxy acids such as lactic acid and glycolic acid, and copolymers with PEG, polyanhydrides, poly(ortho)esters, polyurethanes, poly(butyric acid), poly(valeric acid), poly(lactide-co-caprolactone), blends and copolymers thereof.

Examples of preferred natural polymers include proteins such as albumin and prolamines, for example, zein, and polysaccharides such as alginate, cellulose and polyhydroxyalkanoates, for example, polyhydroxybutyrate.

The in vivo stability of the matrix can be adjusted during the production by using polymers such as polylactide co glycolide copolymerized with polyethylene glycol (PEG). PEG if exposed on the external surface may elongate the time these materials circulate since it is hydrophilic.

Examples of preferred non-biodegradable polymers include ethylene vinyl acetate, poly(meth)acrylic acid, polyamides, copolymers and mixtures thereof.

Particles having an average particle size of between 10 nm and 1000 microns are useful in the compositions described herein. In preferred embodiments, the particles have an average particle size of between 10 nm and 100 microns, more preferably between about 100 nm and about 50 microns, more preferably between about 200 nm and about 50 microns. In certain embodiments, the particles are nanoparticles having a diameter of between 500 and 700 nm. The particles can have any shape but are generally spherical in shape.

C. Implants Formed from Polymer-Drug Conjugates

Implants can be formed from one or more polymer-drug conjugates. In preferred embodiments, the implants are intraocular implants. Suitable implants include, but are not limited to, rods, discs, wafers, and the like.

In some cases, the implants are formed from a single polymer-drug conjugate (i.e., the implants are formed from a polymer-drug conjugate which contains the same active agent, hydrophobic polymer segment, branch point (when present), and hydrophilic polymer segment or segments).

In other embodiments, the implants are formed from a mixture of two or more different polymer-drug conjugates. For example, the implants are formed from two or more polymer-drug conjugates containing one or more anti-glaucoma agents, particularly those agents that lower intraocular pressure (IOP), such as ethacrynic acid (ECA) or a derivative thereof, and different hydrophobic polymer segments, branch points (when present), and/or hydrophilic polymer segments. Such implants can be used, for example, to vary the release rate of the agent or agents.

Implants can also be formed from a polymeric matrix having one or more anti-glaucoma agents, particularly those agents that lower intraocular pressure (IOP), such as ethacrynic acid (ECA) or a derivative thereof dispersed or encapsulated therein. The matrix can be formed of any of the non-biodegradable or biodegradable polymers described in Section B above, although biodegradable polymers are preferred. The composition of the polymer matrix is selected based on the time required for in vivo stability, i.e. that time required for distribution to the site where delivery is desired, and the time desired for delivery.

Implants can also be formed from blends of polymer-drug conjugates with one or more of the polymers described above.

The implants may be of any geometry such as fibers, sheets, films, microspheres, spheres, circular discs, rods, or plaques. Implant size is determined by factors such as toleration for the implant, location of the implant, size limitations in view of the proposed method of implant insertion, ease of handling, etc.

Where sheets or films are employed, the sheets or films will be in the range of at least about 0.5 mm×0.5 mm, usually about 3 to 10 mm×5 to 10 mm with a thickness of about 0.1 to 1.0 mm for ease of handling. Where fibers are employed, the fiber diameter will generally be in the range of about 0.05 to 3 mm and the fiber length will generally be in the range of about 0.5 to 10 mm.

The size and shape of the implant can also be used to control the rate of release, period of treatment, and drug concentration at the site of implantation. Larger implants will deliver a proportionately larger dose, but depending on the surface to mass ratio, may have a slower release rate. The particular size and geometry of the implant are chosen to suit the site of implantation.

Intraocular implants may be spherical or non-spherical in shape. For spherical-shaped implants, the implant may have a largest dimension (e.g., diameter) between about 5 μm and about 2 mm, or between about 10 μm and about 1 mm for administration with a needle, greater than 1 mm, or greater than 2 mm, such as 3 mm or up to 10 mm, for administration by surgical implantation. If the implant is non-spherical, the implant may have the largest dimension or smallest dimension be from about 5 μm and about 2 mm, or between about 10 μm and about 1 mm for administration with a needle, greater than 1 mm, or greater than 2 mm, such as 3 mm or up to 10 mm, for administration by surgical implantation.

The vitreous chamber in humans is able to accommodate relatively large implants of varying geometries, having lengths of, for example, 1 to 10 mm. The implant may be a cylindrical pellet (e.g., rod) with dimensions of about 2 mm×0.75 mm diameter. The implant may be a cylindrical pellet with a length of about 7 mm to about 10 mm, and a diameter of about 0.75 mm to about 1.5 mm. In certain embodiments, the implant is in the form of an extruded filament with a diameter of about 0.5 mm, a length of about 6 mm, and a weight of approximately 1 mg. In some embodiments, the dimensions are, or are similar to, implants already approved for intraocular injection via needle: diameter of 460 microns and a length of 6 mm and diameter of 370 microns and length of 3.5 mm.

Intraocular implants may also be designed to be least somewhat flexible so as to facilitate both insertion of the implant in the eye, such as in the vitreous, and subsequent accommodation of the implant. The total weight of the implant is usually about 250 to 5000 μg, more preferably about 500-1000 rig. In certain embodiments, the intraocular implant has a mass of about 500 μg, 750 μg, or 1000 μg.

Methods of Manufacture

Implants can be manufactured using any suitable technique known in the art. Examples of suitable techniques for the preparation of implants include solvent evaporation methods, phase separation methods, interfacial methods, molding methods, injection molding methods, extrusion methods, coextrusion methods, carver press method, die cutting methods, heat compression, and combinations thereof. Suitable methods for the manufacture of implants can be selected in view of many factors including the properties of the polymer/polymer segments present in the implant, the properties of the one or more anti-glaucoma agents, particularly those agents that lower intraocular pressure (IOP), such as ethacrynic acid (ECA) or a derivative thereof present in the implant, and the desired shape and size of the implant. Suitable methods for the preparation of implants are described, for example, in U.S. Pat. No. 4,997,652 and U.S. Patent Application Publication No. US 2010/0124565.

In certain cases, extrusion methods may be used to avoid the need for solvents during implant manufacture. When using extrusion methods, the polymer/polymer segments and the agent or agents is chosen so as to be stable at the temperatures required for manufacturing, usually at least about 85° Celsius. However, depending on the nature of the polymeric components and ECA or a derivative thereof, extrusion methods can employ temperatures of about 25° C. to about 150° C., more preferably about 65° C. to about 130° C.

Implants may be coextruded in order to provide a coating covering all or part of the surface of the implant. Such coatings may be erodible or non-erodible, and may be impermeable, semi-permeable, or permeable to the agent or agents, water, or combinations thereof. Such coatings can be used to further control release of the agent or agents from the implant.

Compression methods may be used to make the implants. Compression methods frequently yield implants with faster release rates than extrusion methods. Compression methods may employ pressures of about 50-150 psi, more preferably about 70-80 psi, even more preferably about 76 psi, and use temperatures of about 0° C. to about 115° C., more preferably about 25° C.° C.

### IV. Pharmaceutical Formulations

Pharmaceutical formulations contain one or more species of polymer-drug conjugates in combination with one or more pharmaceutically acceptable excipients. Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS), and may be administered to an individual without causing undesirable biological side effects or unwanted interactions.

In some cases, the pharmaceutical formulation contains only one type of conjugate or polymeric particles for the controlled release of one or more anti-glaucoma agents, particularly those agents that lower intraocular pressure (IOP), such as ethacrynic acid (ECA) or a derivative thereof (e.g., a formulation containing polymer-drug conjugate particles wherein the polymer-drug conjugate particles incorporated into the pharmaceutical formulation have the same composition). In other embodiments, the pharmaceutical formulation contains two or more different type of conjugates or polymeric particles for the controlled release of one or more anti-glaucoma agents, particularly those agents that lower intraocular pressure (IOP), such as ethacrynic acid (ECA) or a derivative thereof (e.g., the pharmaceutical formulation contains two or more populations of polymer-drug conjugate particles, wherein the populations of polymer-drug conjugate particles have different chemical compositions, different average particle sizes, and/or different particle size distributions).

B. Formulations for Ocular Administration

Particles formed from the polymer-drug conjugates will preferably be formulated as a solution or suspension for injection to the eye.

Pharmaceutical formulations for ocular administration are preferably in the form of a sterile aqueous solution or suspension of particles formed from one or more polymer-drug conjugates. Acceptable solvents include, for example, water, Ringer's solution, phosphate buffered saline (PBS), and isotonic sodium chloride solution. The formulation may also be a sterile solution, suspension, or emulsion in a nontoxic, parenterally acceptable diluent or solvent such as 1,3-butanediol.

In some instances, the formulation is distributed or packaged in a liquid form. Alternatively, formulations for ocular administration can be packed as a solid, obtained, for example by lyophilization of a suitable liquid formulation. The solid can be reconstituted with an appropriate carrier or diluent prior to administration.

Solutions, suspensions, or emulsions for ocular administration may be buffered with an effective amount of buffer necessary to maintain a pH suitable for ocular administration. Suitable buffers are well known by those skilled in the art and some examples of useful buffers are acetate, borate, carbonate, citrate, and phosphate buffers.

Solutions, suspensions, or emulsions for ocular administration may also contain one or more tonicity agents to adjust the isotonic range of the formulation. Suitable tonicity agents are well known in the art and some examples include glycerin, mannitol, sorbitol, sodium chloride, and other electrolytes.

Solutions, suspensions, or emulsions for ocular administration may also contain one or more preservatives to prevent bacterial contamination of the ophthalmic preparations. Suitable preservatives are known in the art, and include polyhexamethylenebiguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof.

Solutions, suspensions, or emulsions for ocular administration may also contain one or more excipients known art, such as dispersing agents, wetting agents, and suspending agents.

### V. Methods of Use

A. Diseases and Disorders to be Treated

Controlled release dosage formulations for the delivery of one or more anti-glaucoma agents, particularly those agents that lower intraocular pressure (IOP), such as ethacrynic acid (ECA) or a derivative thereof can be used to treat or a disease or disorder associated with increased intraocular pressure. Upon administration, the agent or agents is released over an extended period of time at concentrations which are high enough to produce therapeutic benefit, but low enough to avoid cytotoxicity.

When administered to the eye, the particles release a low dose of one or more active agents over an extended period of time, preferably longer than 3, 7, 10, 15, 21, 25, 30, or 45 days. The structure of the polymer-drug conjugate or makeup of the polymeric matrix, particle morphology, and dosage of particles administered can be tailored to administer a therapeutically effective amount of one or more active agents to the eye over an extended period of time while minimizing side effects, such as the reduction of scoptopic ERG b-wave amplitudes and/or retinal degeneration.

Pharmaceutical compositions containing particles for the controlled release of one or more anti-glaucoma agents, particularly those agents that lower intraocular pressure (IOP), such as ethacrynic acid (ECA) or a derivative thereof can be administered to the eye of a patient in need thereof to treat or prevent one or more diseases or disorders of the eye. Typically, the conjugate is administered to the anterior chamber, trabecular meshwork, and Schlemms canal.

In preferred embodiments, a pharmaceutical composition containing particles formed from one or more of the polymer-drug conjugates provided herein is administered to treat or prevent an intraocular neovascular disease. In certain embodiments, the particles are formed from a polymer-drug conjugate containing an anthracycline, such as daunorubicin or doxorubicin.

Eye diseases, particularly those characterized by ocular neovascularization, represent a significant public health concern. Intraocular neovascular diseases are characterized by unchecked vascular growth in one or more regions of the eye. Unchecked, the vascularization damages and/or obscures one or more structures in the eye, resulting in vision loss. Intraocular neovascular diseases include proliferative retinopathies, choroidal neovascularization (CNV), age-related macular degeneration (AMD), diabetic and other ischemia-related retinopathies, diabetic macular edema, pathological myopia, von Hippel-Lindau disease, histoplasmosis of the eye, central retinal vein occlusion (CRVO), corneal neovascularization, and retinal neovascularization (RNV). Intraocular neovascular diseases afflict millions worldwide, in many cases leading to severe vision loss and a decrease in quality of life and productivity.

Age related macular degeneration (AMD) is a leading cause of severe, irreversible vision loss among the elderly. Bressler, et al. JAMA, 291:1900-1901 (2004). AMD is characterized by a broad spectrum of clinical and pathologic findings, such as pale yellow spots known as drusen, disruption of the retinal pigment epithelium (RPE), choroidal neovascularization (CNV), and disciform macular degeneration. AMD is classified as either dry (i.e., non-exudative) or wet (i.e., exudative). Dry AMD is characterized by the presence of lesions called drusen. Wet AMD is characterized by neovascularization in the center of the visual field.

Although less common, wet AMID is responsible for 80%-90% of the severe visual loss associated with AMD (Ferris, et al. Arch. Ophthamol. 102:1640-2 (1984)). The cause of AMD is unknown. However, it is clear that the risk of developing AMD increases with advancing age. AMD has also been linked to risk factors including family history, cigarette smoking, oxidative stress, diabetes, alcohol intake, and sunlight exposure.

Wet AMD is typically characterized by CNV of the macular region. The choroidal capillaries proliferate and penetrate Bruch's membrane to reach the retinal pigment epithelium (RPE). In some cases, the capillaries may extend into the subretinal space. The increased permeability of the newly formed capillaries leads to accumulation of serous fluid or blood under the RPE and/or under or within the neurosensory retina. Decreases in vision occur when the fovea becomes swollen or detached. Fibrous metaplasia and organization may ensue, resulting in an elevated subretinal mass called a disciform scar that constitutes end-stage AMD and is associated with permanent vision loss (D'Amico D J. N. Engl. J. Med. 331:95-106 (1994)).

Other diseases and disorders of the eye, such as uveitis, are also difficult to treat using existing therapies. Uveitis is a general term referring to inflammation of any component of the uveal tract, such as the iris, ciliary body, or choroid. Inflammation of the overlying retina, called retinitis, or of the optic nerve, called optic neuritis, may occur with or without accompanying uveitis.

Ocular complications of uveitis may produce profound and irreversible loss of vision, especially when unrecognized or treated improperly. The most frequent complications of uveitis include retinal detachment, neovascularization of the retina, optic nerve, or iris, and cystoid macular edema. Macular edema (ME) can occur if the swelling, leaking, and background diabetic retinopathy (BDR) occur within the macula, the central 5% of the retina most critical to vision. ME is a common cause of severe visual impairment.

There have been many attempts to treat intraocular neurovascular diseases, as well as diseases associated with chronic inflammation of the eye, with pharmaceuticals. Attempts to develop clinically useful therapies have been plagued by difficulty in administering and maintaining a therapeutically effective amount of the pharmaceutical in the ocular tissue for an extended period of time. In addition, many pharmaceuticals exhibit significant side effects and/or toxicity when administered to the ocular tissue.

Intraocular neovascular diseases are diseases or disorders of the eye that are characterized by ocular neovascularization. The neovascularization may occur in one or more regions of the eye, including the cornea, retina, choroid layer, or iris. In certain instances, the disease or disorder of the eye is characterized by the formation of new blood vessels in the choroid layer of the eye (i.e., choroidal neovascularization, CNV). In some instances, the disease or disorder of the eye is characterized by the formation of blood vessels originating from the retinal veins and extending along the inner (vitreal) surface of the retina (i.e., retinal neovascularization, RNV).

Exemplary neovascular diseases of the eye include age-related macular degeneration associated with choroidal neovascularization, proliferative diabetic retinopathy (diabetic retinopathy associated with retinal, preretinal, or iris neovascularization), proliferative vitreoretinopathy, retinopathy of prematurity, pathological myopia, von Hippel-Lindau disease, presumed ocular histoplasmosis syndrome (POHS), and conditions associated with ischemia such as branch retinal vein occlusion, central retinal vein occlusion, branch retinal artery occlusion, and central retinal artery occlusion.

The neovascularization can be caused by a tumor. The tumor may be either a benign or malignant tumor. Exemplary benign tumors include hamartomas and neurofibromas. Exemplary malignant tumors include choroidal melanoma, uveal melanoma or the iris, uveal melanoma of the ciliary body, retinoblastoma, or metastatic disease (e.g., choroidal metastasis).

The neovascularization may be associated with an ocular wound. For example, the wound may the result of a traumatic injury to the globe, such as a corneal laceration. Alternatively, the wound may be the result of ophthalmic surgery.

The polymer-drug conjugates can be administered to prevent or reduce the risk of proliferative vitreoretinopathy following vitreoretinal surgery, prevent corneal haze following corneal surgery (such as corneal transplantation and eximer laser surgery), prevent closure of a trabeculectomy, or to prevent or substantially slow the recurrence of pterygii.

The polymer-drug conjugates can be administered to treat or prevent an eye disease associated with inflammation. In such cases, the polymer-drug conjugate preferably contains an anti-inflammatory agent. Exemplary inflammatory eye diseases include, but are not limited to, uveitis, endophthalmitis, and ophthalmic trauma or surgery.

The eye disease may also be an infectious eye disease, such as HIV retinopathy, toxocariasis, toxoplasmosis, and endophthalmitis.

Pharmaceutical compositions containing particles formed from one or more of the polymer-drug conjugates can also be used to treat or prevent one or more diseases that affect other parts of the eye, such as dry eye, meibomitis, glaucoma, conjunctivitis (e.g., allergic conjunctivitis, vernal conjunctivitis, giant papillary conjunctivitis, atopic keratoconjunctivitis), neovascular glaucoma with iris neovascularization, and iritis.

B. Methods of Administration

The formulations can be administered locally to the eye by intravitreal injection (e.g., front, mid or back vitreal injection), subconjunctival injection, intracameral injection, injection into the anterior chamber via the temporal limbus, intrastromal injection, injection into the subchoroidal space, intracorneal injection, subretinal injection, and intraocular injection. In a preferred embodiment, the pharmaceutical composition is administered by intravitreal injection.

The implants can be administered to the eye using suitable methods for implantation known in the art. In certain embodiments, the implants are injected intravitreally using a needle, such as a 22-guage needle. Placement of the implant intravitreally may be varied in view of the implant size, implant shape, and the disease or disorder to be treated.

In some embodiments, the pharmaceutical compositions and/or implants described herein are co-administered with one or more additional active agents. “Co-administration”, as used herein, refers to administration of the controlled release formulation of ECA or a derivative thereof with one or more additional active agents within the same dosage form, as well as administration using different dosage forms simultaneously or as essentially the same time. “Essentially at the same time” as used herein generally means within ten minutes, preferably within five minutes, more preferably within two minutes, most preferably within in one minute.

In some embodiments, the pharmaceutical compositions and/or implants described herein are co-administered with one or more additional treatments for a neovascular disease or disorder of the eye. In some embodiments, the pharmaceutical compositions and/or implants described herein are co-administered with one or more anti-angiogenesis agent such bevacizumab (AVASTIN®), ranibizumab, LUCENTIS®, or aflibercept (EYLEA®).

Preferably, the particles will release an effective amount of one or more anti-glaucoma agents, particularly those agents that lower intraocular pressure (IOP), such as ethacrynic acid (ECA) or a derivative thereof over an extended period of time. In preferred embodiments, the particles release an effective amount of the agent or agents over a period of at least two weeks, more preferably over a period of at least four weeks, more preferably over a period of at least six weeks, most preferably over a period of at least eight weeks. In some embodiments, the particles release an effective amount of the agent or agents over a period of three months or longer.

Generally, the therapeutic efficacy of the compositions described herein is characterized by lowering of the IOP relative to an IOP of an eye without any treatment or to an IOP of an eye receiving vehicle or control substance (control). Typically, the lowering of the IOP relative to that of a control is lowering by 1-8 mmHg, preferably by 2-6 mmHg, and more preferably by 2-4 mmHg.

The lowering of the IOP occurs over a prolonged period of time, typically ranging from two to seven days to one to six months or more. Preferably, the reduction in IOP occurs within days and remains lower than that in the control for a period of one to six months, more preferably for a period of three to four months.

The present invention will be further understood by reference to the following non-limiting examples.

### Examples

### Example 1. Preparation of ECA-L-Cysteine

100 mg ethacrynic acid (ECA) was added to 3 mL water and the pH was adjusted to 5.0 until the ECA was dissolved. After dissolution, the pH was adjusted to 7.0. 39 mg L-cysteine was dissolved in 3 ml double-distilled water and the pH was adjusted to 7.0. The two solutions were mixed with gentle rolling for 1 hour, after which the solution was lyophilized.

### Example 2. Preparation of the PEG3-PSA (PEG-PSA) Prepolymer

(Polyethylene glycol)₃-co-poly(sebacic acid) (PEG3-PSA) or (Polyethylene glycol)-co-poly(sebacic acid) (PEG-PSA) was synthesized by melt condensation. Sebacic acid (SA) was refluxed in acetic anhydride to form a sebacic acid (SA) prepolymer (Acyl-SA). Polyethylene glycol (PEG₃) was prepared by mixing CH₃O-PEG-NH₂ (2.0 g), citric acid (26 g), dicyclohexylcarbodiimide (DCC; 83 mg) and 4-(dimethylamino)pyridine (DMAP, 4.0 mg) which were added to 10 mL methylene chloride, stirred overnight at room temperature, then precipitated and washed with ether, and dried under vacuum. Acyl-SA (90% w/v) and PEG₃ ((10% w/v) (or PEG) were polymerized at 180° C. for 30 minutes. Throughout the polymerization, a strong nitrogen sweep was performed for 30 sec every 15 min. At the end of the reaction, the polymers were allowed to cool completely and dissolved in chloroform. The solution was precipitated dropwise into excess petroleum ether. The precipitate was collected by filtration and dried under vacuum to constant weight.

### Example 3. Preparation of ECA-L-Cysteine Polyanhydride Microspheres

120 mg of PEG-PSA or PEG3-SA was dissolved in 1.2 mL of dichloromethane and 30 mg of ECA-L-cysteine in 1.2 mL of DCM, 300 ul methanol and 300 ul DMSO. The two solutions were mixed together and stirred for one hour and poured into a 40 mL 1% Polyvinyl alcohol (PVA, 250000 Mw, 88% hydrolyzed, Sigma) solution, homogenized 1 min (Silverson Homogenizer, model L4RT, Chesham Bucks, England) at 3500 rpm, and then stirred for 3 hours for dichloromethane to evaporate.

The structure of PEG-SA-ECA-L-Cysteine polymer was verified by ¹H NMR using a Bruker Avance 500 MHz FT-NMR spectrometer (Madison, Wis.) and Fourier transform infrared spectroscopy (FT-IR) using a Perkin Elmer 1600 series Fourier transform infrared spectrometer (KBr plate) (Wellesley, Mass.).

The particles were collected by centrifugation and washed in distilled water. Microparticle size analysis was performed with a Coulter Multisizer e (Beckman-Coulter Inc., Fullerton, Calif.). The microparticles were added to 100 mL of Isoton II solution until the coincidence of particles was between 8% and 10%. Greater than 100,000 particles were sized for each batch of microparticles to determine the mean particle size and size distribution. ECA-cysteine particles displayed a particle size of 9.1+3.5 um with a drug loading of 10.2% (weight of drug/total weight).

### Example 4: Determination of Release Kinetics and In Vivo Efficacy

Materials and Methods

Previous studies have demonstrated that intracameral administration of ECA in human patients with elevated IOP resulted in IOP lowering from 3 to 24 hours after ECA treatment, lasting for three days with a gradual return of IOP to pretreatment levels one week after treatment. To evaluate the IOP lowering effect of the PEG-SA-ECA-L-Cysteine particles in normal mice, ECA (1 μg of free drug) or PEG-SA-ECA-L-Cysteine particles (1 μg active drug agent) were administered to normal, C57BL/6 mice via the epischleral vein at the limbus.

Results

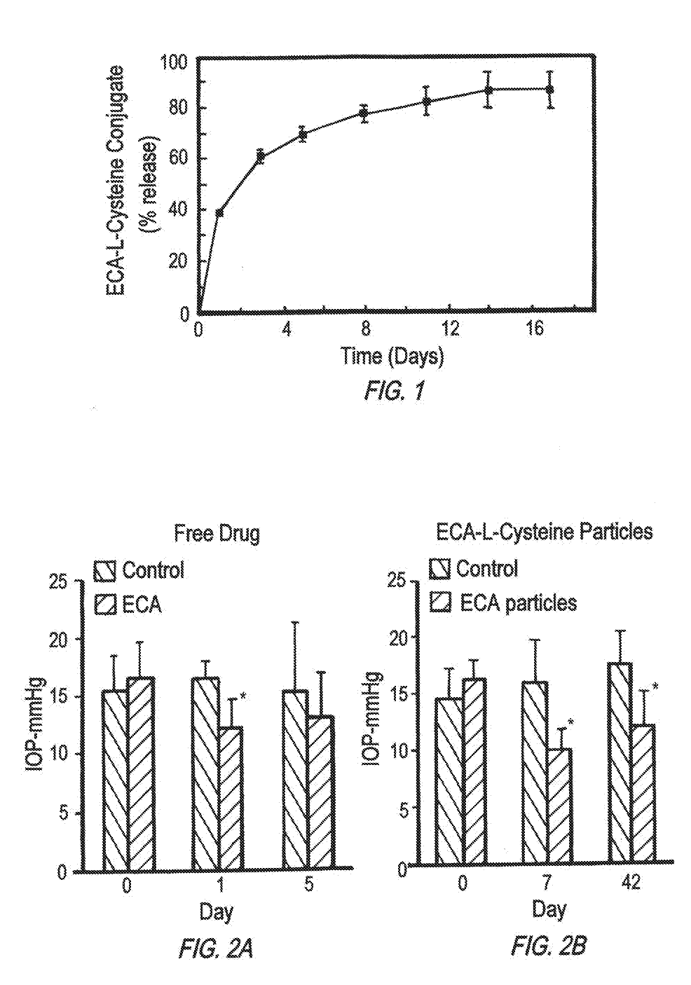

In vitro drug release kinetics under accelerated infinite sink conditions at 37° C. demonstrated that ECA-L-Cysteine conjugate was continually released for 14 days, as shown in FIG. 1.

Treatment with free ECA resulted in a significant lowering of IOP compared to the untreated control group, as shown in FIGS. 2A and 2B. However, this effect was sustained for only 1 day. By Day 5 the IOP lowering effect of free ECA was gone. In contrast, administration of the PEG-SA-ECA-L-Cysteine particles resulted in a sustained IOP lowering effect that lasted for at least 42 days. These data indicate that ECA significantly lowers IOP in normal mice and that the PEG-SA-ECA-L-Cysteine particles markedly prolong the IOP lowering effect of ECA.

1. A polymeric conjugate defined by one of the following formulae
(A-X)ₘ—Y—((Z)ₒ—(X)ₚ-(A)_{q})ₙ
wherein
A represents, independently for each occurrence, one or more anti-glaucoma agents;
X represents, independently for each occurrence, a hydrophobic polymer segment;
Y is absent, or represents a branch point;
Z represents, independently for each occurrence, a hydrophilic polymer segment; and
o, p, and q are independent 0 or 1;
m is an integer between one and twenty; and
n is an integer between zero and twenty, with the proviso that A is not doxorubicin when m and n are both equal to one.

2. The polymer conjugate of claim 1, wherein the A is an anti-glaucoma agent that lowers intraocular pressure (IOP).

3. The polymer conjugate of claim 2, wherein the agent acts directly on the trabecular meshwork (TM).

4. The polymeric conjugate of claim 3, wherein A is ethacrynic acid L-cysteine adduct (ECA-L-cysteine).

5. The polymeric conjugate of claim 1, wherein Z is selected from the group consisting of a poly(alkylene glycol), a polysaccharide, poly(vinyl alcohol), polypyrrolidone, a polyoxyethylene block copolymer (PLURONIC®), and copolymers thereof.

6. The polymeric conjugate of claim 5, wherein Z for each occurrence comprises polyethylene glycol.

7. The polymeric conjugate of claim 1, wherein X is biodegradable.

8. The polymeric conjugate of claim 7, wherein X is selected from the group consisting of polyesters, polycaprolactone, polyanhydrides, and copolymers thereof.

9. The polymeric conjugate of claim 8, wherein X comprises a polyanhydride.

10. The polymeric conjugate of claim 9, wherein X comprises polysebacic anhydride.

11. The polymeric conjugate of claim 9, wherein X comprises 1,6 bis(p-carboxyphenoxy)hexane (CPH) or a combination of poly-CPH (PCPH) and polysebacic anhydride.

12. The polymeric conjugate of claim 1, wherein Y is one of the following:

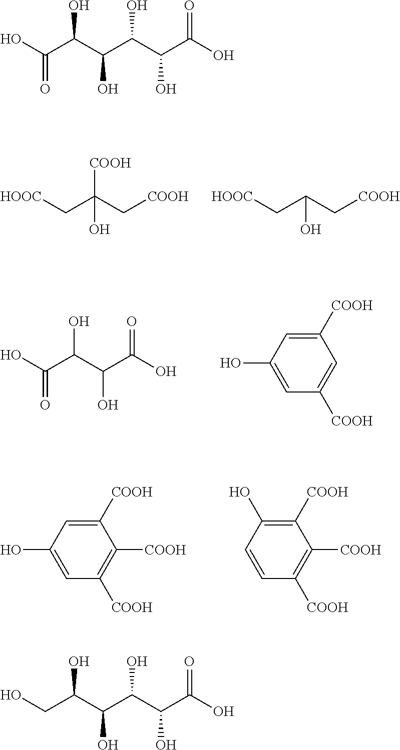

13. The polymeric conjugate of claim 12, wherein Y is citric acid.

14. The polymeric conjugate of claim 1, defined by the following formula
A-X—YZ)ₙ
wherein n is an integer between one and ten or two and ten.

15. The polymeric conjugate of claim 14, wherein n is between 2 and 6.

16. The polymeric conjugate of claim 14, wherein n is 3.

17. The polymeric conjugate of claim 1, wherein the polymeric conjugate is defined by Formula I 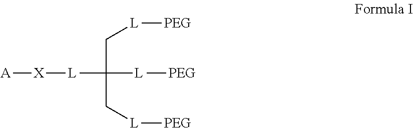
wherein
L represents, independently for each occurrence, an ether (—O—), thioether (—S—), secondary amine (—NH—), tertiary amine (—NR—), secondary amide (—NHCO—; —CONH—), tertiary amide (—NRCO—; —CONR—), secondary carbamate (—OCONH—; —NHCOO—), tertiary carbamate (—OCONR—; —NRCOO—), urea (—NHCONH—; —NRCONH—; —NHCONR—, —NRCONR—), sulfinyl group (—SO—), or sulfonyl group (—SOO—);
R is, individually for each occurrence, an alkyl, cycloalkyl, heterocycloalkyl, alkylaryl, alkenyl, alkynyl, aryl, or heteroaryl group, optionally substituted with between one and five substituents individually selected from alkyl, cyclopropyl, cyclobutyl ether, amine, halogen, hydroxyl, ether, nitrile, CF₃, ester, amide, urea, carbamate, thioether, carboxylic acid, and aryl; and
PEG represents a polyethylene glycol chain.

18. The polymeric conjugate of claim 17, wherein one or more of L are amides or esters.

19. The polymeric conjugate of claim 1, wherein the polymeric conjugate is defined by Formula IA 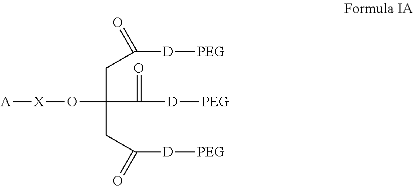
wherein
D represents, independently for each occurrence, O or NH; and
PEG represents a polyethylene glycol chain.

20. A population of micro- and/or nanoparticles comprising the conjugates of claim 1.

21. A formulation comprising the particles of claim 20 in a pharmaceutically acceptable carrier, matrix, hydrogel or implant.

22. A method of treating a disease or disorder of the eye comprising administering to the eye of a patient in need thereof, the particles of claim 20 in a pharmaceutically acceptable carrier, matrix, hydrogel or implant for administration to the eye.
